(19) United States
(12) Patent Application Publication
(10) Pub. No.: US 2017/0109495 A1
(43) Pub. Date: Apr. 20, 2017

(54) Method for measuring blood pressure and embedded device for implementing the same

(71) Applicant: Qin XIN, Beijing (CN)

(72) Inventor: Qin XIN, Beijing (CN)

(21) Appl. No.: 15/308,410

(22) PCT Filed: Jan. 15, 2015
(86) PCT No.: PCT/CN2015/070725

§ 371 Date: Nov. 2, 2016

(30) Foreign Application Priority Data
Apr. 22, 2014 (CN) 201410163425.4

(51) Int. Cl. G06F 19/00; A61B 5/00; G06F 17/11; A61B 5/021

(52) U.S. Cl. CPC G06F 19/3437; A61B 5/02108; A61B 5/681; A61B 5/0064; G06F 17/11; G06F 19/345

(57) ABSTRACT

The present invention provides a method for measuring blood pressure, the method comprising: obtaining a pulse waveform of an measured object, and extracting a plurality of characteristic points from the pulse waveform according to a preset rule; selecting and loading a best blood pressure measurement model group from a model library according to a physiological index of the measured object; and operating the best blood pressure measurement model group to obtain blood pressure parameters of the measured object by calculating according to the plurality of characteristic points. Correspondingly, the present invention further provides an embedded device that may implement the above method for measuring blood pressure. The present invention can, according to measured objects of different types, correspondingly select the best blood pressure measurement model group that is suitable for the measured object, so as to obtain the blood pressure parameters that are more precise.

### TECHNICAL FIELD

The present invention relates to the field of medical measuring instruments, and in particular to a method for measuring blood pressure and an embedded device for implementing the method.

### BACKGROUND ART

The physiological parameters of the human body are a series of indices that medical science uses to assess the physiological state of the human body; these include pulse parameters, blood pressure parameters, blood oxygen parameters, blood sugar parameters and so on. The physiological parameters reflect the condition of the human body in a macroscopic respect, and have very important warning and directing functions for disease prediction and body maintenance. As such, regarding the measurement of blood pressure parameters, the following two modes are mainly employed in the prior art: one is measuring blood pressure parameters by using pressure sphygmomanometer, and another is measuring blood pressure parameters by using pulse wave conducting duration.

Although people can measure and obtain their own blood pressure parameters by employing the above two modes of blood pressure measurement, both of these two modes of blood pressure measurement have certain drawbacks. Regarding the first mode, measuring blood pressure parameters by using a pressure sphygmomanometer tends to tremendously disturb human body, and cannot achieve the aim of continuous measurement. Regarding the second mode, measuring blood pressure by using pulse wave conducting duration has a large error, and cannot simultaneously measure the systolic pressure. Therefore, it is desired to develop a method for measuring blood pressure and a corresponding measuring device that can solve the above drawbacks.

### SUMMARY OF THE INVENTION

In order to overcome the above drawbacks of the prior art, the present invention provides a method for measuring blood pressure, the method comprising:

obtaining a pulse waveform of an measured object, and extracting a plurality of characteristic points from the pulse waveform according to a preset rule;

selecting and loading a best blood pressure measurement model group from a model library according to a physiological index of the measured object; and

operating the best blood pressure measurement model group to obtain blood pressure parameters of the measured object by calculating according to the plurality of characteristic points.

According to one aspect of the present invention, in the method, the obtaining a pulse waveform of an measured object comprises: sending a measuring light of at least one wavelength to a body surface skin of the measured object, and receiving a reflected light of the measuring light; and processing the reflected light to obtain the pulse waveform of the measured object.

According to another aspect of the present invention, in the method, the body surface skin is a wrist body surface skin corresponding to a radial artery of the measured object.

According to yet another aspect of the present invention, in the method, the measuring light of at least one wavelength comprises red light and/or infrared light.

According to yet another aspect of the present invention, in the method, a range of a wavelength of the red light is 660 nm±3 nm; and a range of a wavelength of the infrared light is 940 nm±10 nm.

According to yet another aspect of the present invention, in the method, the characteristic points comprise a pulse frequency, an area of wave pattern of photoplethysmography, an area of wave pattern of principal wave upstroke, a stroke volume, a waveform factor of pulse wave, an upstroke area ratio, an average gradient of ascending limb, a relative height of a dicrotic notch, and a relative height of a dicrotic wave.

According to yet another aspect of the present invention, in the method, the selecting and loading a best blood pressure measurement model group from a model library according to a physiological index of the measured object comprises: if it is determined according to the physiological index of the measured object that the measured object is a youth, the best blood pressure measurement model group comprises a youth diastolic pressure measurement model and a youth systolic pressure measurement model; if it is determined according to the physiological index of the measured object that the measured object is a middle-aged adult, the best blood pressure measurement model group comprises a middle-aged adult diastolic pressure measurement model and a middle-aged adult systolic pressure measurement model, the middle-aged adult systolic pressure measurement model comprising a middle-aged adult reference measurement submodel, a middle-aged adult normal measurement submodel, and a middle-aged adult hypertension measurement submodel; and if it is determined according to the physiological index of the measured object that the measured object is an aged person, the best blood pressure measurement model group comprises an aged person diastolic pressure measurement model and an aged person systolic pressure measurement model, the aged person systolic pressure measurement model comprising an aged person reference measurement submodel, an aged person normal measurement submodel, and an aged person hypertension measurement submodel.

According to yet another aspect of the present invention, in the method, the operating the best blood pressure measurement model group to obtain blood pressure parameters of the measured object by calculating according to the plurality of characteristic points comprises: the measured object being a middle-aged adult; substituting the plurality of characteristic points into the middle-aged adult diastolic pressure measurement model, and obtaining a numerical value of the diastolic pressure of the blood pressure parameters by calculating; and substituting the plurality of characteristic points into the middle-aged adult reference measurement submodel, the middle-aged adult normal measurement submodel and the middle-aged adult hypertension measurement submodel, obtaining respectively a first numerical value, a second numerical value, and a third numerical value by calculating, and selecting the numerical value closest to the first numerical value from the second numerical value and the third numerical value as a numerical value of the systolic pressure of the blood pressure parameters.

According to yet another aspect of the present invention, in the method, the operating the best blood pressure measurement model group to obtain blood pressure parameters of the measured object by calculating according to the plurality of characteristic points comprises: the measured object being an aged person; substituting the plurality of characteristic points into the aged person diastolic pressure measurement model, and obtaining a numerical value of the diastolic pressure of the blood pressure parameters by calculating; and substituting the plurality of characteristic points into the aged person reference measurement submodel, the aged person normal measurement submodel and the aged person hypertension measurement submodel, obtaining respectively a fourth numerical value, a fifth numerical value and a sixth numerical value by calculating, and selecting the numerical value closest to the fourth numerical value from the fifth numerical value and the sixth numerical value as a numerical value of the systolic pressure of the blood pressure parameters.

The present invention further provides an embedded device for implementing the above method for measuring blood pressure, the embedded device comprising:

an obtaining module, for obtaining the pulse waveform; and

a processing module, for extracting the plurality of characteristic points from the pulse waveform according to the preset rule, selecting and loading the best blood pressure measurement model group from the model library according to a physiological index of the measured object, and operating the best blood pressure measurement model group to obtain the blood pressure parameters by calculating according to the plurality of characteristic points.

According to one aspect of the present invention, the embedded device is integrated on portable equipment, and the portable equipment has a wrist-worn structure.

The method for measuring blood pressure and the embedded device for implementing the method provided by the present invention have the following advantages:

Firstly, compared with the conventional mode of measuring blood pressure by using a pressure sphygmomanometer, the present invention measures blood pressure by using the characteristic points of the pulse waveform, which does not disturb human body and can realize the continuous measurement on blood pressure parameters. Compared with the conventional mode of measuring blood pressure by using pulse conducting duration, the present invention measures blood pressure by using the characteristic points of the pulse waveform, which can obtain the blood pressure parameters of the measured object that are more precise.

Secondly, the method, according to the physiological index of the measured object, correspondingly selects the best blood pressure measurement model group as to the measured object, whereby the accuracy of blood pressure parameter measurement can be further improved.

### BRIEF DESCRIPTION OF DRAWINGS

The other features, objects and advantages of the present invention will become more apparent by reading the detailed description of non-limiting embodiments made with reference to the following attached figures:

FIG. 1 is the flow chart of a specific embodiment of the method for measuring blood pressure according to the present invention;

FIG. 2 is the structural schematic representation of a specific embodiment of the embedded device for implementing the method for measuring blood pressure according to the present invention;

FIG. 3 is the structural schematic representation of a preferable embodiment of portable equipment that integrates the embedded device for implementing the method for measuring blood pressure and has a wrist-worn structure according to the present invention; and

FIG. 4 is the structural schematic representation of another preferable embodiment of portable equipment that integrates the embedded device for implementing the method for measuring blood pressure and has a wrist-worn structure according to the present invention.

The same or similar reference elements in the attached figures represent the same or similar components.

### SPECIFIC EMBODIMENTS

In order to better understand and explain the present invention, the present invention will be further described in detail below with reference to the attached figures.

Before the detailed description of the present invention, it should be noted that, the primary application object of the method and system for measuring blood pressure provided by the present invention is a human being, and thus the measured object herein mainly refers to the persons that need blood pressure measurement. A person skilled in the art should understand that the method and device for measuring blood pressure provided by the present invention may also be applied to blood pressure measurement targeting mammals that have physiological characteristics the same as or similar to those of human beings.

The present invention provides a method for measuring blood pressure. Referring to FIG. 1, FIG. 1 is the flow chart of a specific embodiment of the method for measuring blood pressure according to the present invention. As shown in the figure, the method for measuring blood pressure comprises:

In Step S101, obtaining a pulse waveform of a measured object, and extracting a plurality of characteristic points from the pulse waveform according to a preset rule;

In Step S102, selecting and loading a best blood pressure measurement model group from a model library according to a physiological index of the measured object;

In Step S103, operating the best blood pressure measurement model group to obtain blood pressure parameters of the measured object by calculating according to the plurality of characteristic points.

Specifically, in Step S101, first, the method sends a measuring light of at least one wavelength to a body surface skin of the measured object, and receives a reflected light of the measuring light. In the present embodiment, the body surface skin is a wrist body surface skin corresponding to a radial artery of the measured object. The measuring light of at least one wavelength comprises red light and/or infrared light. As such, a range of a wavelength of the red light is 660 nm±3 nm, and a range of a wavelength of the infrared light is 940 nm±10 nm. Then, the method processes the received reflected light to obtain the pulse waveform of the measured object. Then, the method extracts a plurality of characteristic points from the pulse waveform according to a preset rule, wherein, the plurality of characteristic points are used for calculating blood pressure parameters of the measured object. In the present embodiment, the characteristic points comprise a pulse frequency, an area of wave pattern of photoplethysmography, an area of wave pattern of principal wave upstroke, stroke volume, a waveform factor of pulse wave, upstroke area ratio, an average gradient of ascending limb, a relative height of dicrotic notch, and a relative height of dicrotic wave. In order that the measurement on the blood pressure parameters is more precise, in other embodiments, the characteristic points may further comprise a principal wave height, a dicrotic wave height, a dicrotic notch height, a baseline height, a principal wave rise time, a systole duration, a diastole duration, a mean area of unit time, and a duration ratio of systole to diastole.

It should be noted that, by utilizing reflection principles, obtaining a pulse waveform of a measured object and extracting characteristic points from the pulse waveform according to a preset rule are technical means that are well known by a person skilled in the art, and for the sake of conciseness, the processes of them will not be described in detail here.

In Step S102, the method classifies the measured object according to the physiological index of the measured object, and, after the classifying, selects and loads from a model library a best blood pressure measurement model group regarding the measured object of the class. As such, the best blood pressure measurement model group is used for calculating blood pressure parameters of the measured object, and the blood pressure parameters comprise the diastolic pressure numerical value and the systolic pressure numerical value of the measured object. In the present embodiment, the physiological index that is used for classifying the measured object is age. Preferably, the method may, according to the age classifying criteria of China, define the population of the ages of 18 to 40 as youth, define the population of the ages of 41 to 65 as middle-aged adult, and define the population of the ages above 66 as aged person.

If the measured object is a youth, the best blood pressure measurement model group suitable for the measured object comprises a youth diastolic pressure measurement model and a youth systolic pressure measurement model.

If the measured object is a middle-aged adult, the best blood pressure measurement model group suitable for the measured object comprises a middle-aged adult diastolic pressure measurement model and a middle-aged adult systolic pressure measurement model, wherein the middle-aged adult systolic pressure measurement model comprises a middle-aged adult reference measurement submodel, a middle-aged adult normal measurement submodel and a middle-aged adult hypertension measurement submodel.

If the measured object is an aged person, the best blood pressure measurement model group suitable for the measured object comprises an aged person diastolic pressure measurement model and an aged person systolic pressure measurement model, wherein the aged person systolic pressure measurement model comprises an aged person reference measurement submodel, an aged person normal measurement submodel and an aged person hypertension measurement submodel.

In the present embodiment, the best blood pressure measurement model group comprises a regression equation, wherein a regression coefficient of the regression equation is generated according to a statistical treatment regarding a sample set. The following description will take the best blood pressure measurement model group suitable for youth as an example. The youth diastolic pressure measurement model comprises a regression equation that is suitable for calculating the diastolic pressure numerical value of youth, and the youth systolic pressure measurement model comprises a regression equation that is suitable for calculating the systolic pressure numerical value of youth. The particular values of the regression coefficients of the above two regression equations can be obtained according to the statistical treatment on the pulse waveform characteristic points and the blood pressure parameters of each of the samples in a sample set (for example, a sample set comprising 100 samples) of youths.

Furthermore, a person skilled in the art can understand that, the physiological index of the measured object is not limited to age, and all the physiological indexes that can be used for classifying the measured object (provided that each class has a corresponding blood pressure measurement model) are included in the protection scope of the present invention. For the sake of conciseness, the physiological indexes will not be in detail enumerated herein.

In Step S103, specifically described is how to obtain the diastolic pressure numerical value and the systolic pressure numerical value of a measured object by a best blood pressure measurement model by calculating individually regarding the three cases that the measured object is a youth, a middle-aged adult or an aged person.

If the measured object is a youth, the method substitutes the plurality of characteristic points extracted from the pulse waveform of the measured object into the youth diastolic pressure measurement model and the youth systolic pressure measurement model, and obtains the diastolic pressure numerical value and the systolic pressure numerical value of the measured object by calculating respectively.

If the measured object is a middle-aged adult, the method substitutes the plurality of characteristic points extracted from the pulse waveform of the measured object into the middle-aged adult diastolic pressure measurement model and the middle-aged adult systolic pressure measurement model, and obtains the diastolic pressure numerical value and the systolic pressure numerical value of the measured object by calculating respectively. As such, the process of calculating the systolic pressure numerical value by the middle-aged adult systolic pressure measurement model is as follows: first, substituting the plurality of characteristic points into the middle-aged adult reference measurement submodel, the middle-aged adult normal measurement submodel, and the middle-aged adult hypertension measurement submodel, wherein three numerical values can be obtained when calculating by operating each of the submodels, which are respectively a first numerical value, a second numerical value and a third numerical value; and then, selecting the numerical value closest to the first numerical value from the second numerical value and the third numerical value as the systolic pressure numerical value of the measured object; that is, comparing the absolute value of the difference value between the first numerical value and the second numerical value and the absolute value of the difference value between the first numerical value and the third numerical value, and if the absolute value of the difference value between the first numerical value and the second numerical value is less than the absolute value of the difference value between the first numerical value and the third numerical value, determining that the measured object belongs to the middle-aged adult normal population, and in this case taking the second numerical value that is outputted by the middle-aged adult normal measurement submodel as the systolic pressure numerical value of the measured object, and if the absolute value of the difference value between the first numerical value and the second numerical value is greater than the absolute value of the difference value between the first numerical value and the third numerical value, determining that the measured object belongs to the middle-aged adult hypertension population, and in this case taking the third numerical value that is outputted by the middle-aged adult hypertension measurement submodel as the systolic pressure numerical value of the measured object.

When the measured object is an aged person, the method is similar to the case when the measured object is a middle-aged adult. Specifically, if the measured object is an aged person, the method substitutes the plurality of characteristic points extracted from the pulse waveform of the measured object into the aged person diastolic pressure measurement model and the aged person systolic pressure measurement model, and obtains the diastolic pressure numerical value and the systolic pressure numerical value of the measured object by calculating, respectively. As such, the process of calculating the systolic pressure numerical value by the aged person systolic pressure measurement model is as follows: first, substituting the plurality of characteristic points into the aged person reference measurement submodel, the aged person normal measurement submodel, and the aged person hypertension measurement submodel, wherein three numerical values can be obtained when calculating by operating each of the submodels, which are respectively a fourth numerical value, a fifth numerical value and a sixth numerical value; and then, selecting the numerical value the closest to the fourth numerical value from the fifth numerical value and the sixth numerical value as the systolic pressure numerical value of the measured object; that is, comparing the absolute value of the difference value between the fourth numerical value and the fifth numerical value and the absolute value of the difference value between the fourth numerical value and the sixth numerical value, and if the absolute value of the difference value between the fourth numerical value and the fifth numerical value is less than the absolute value of the difference value between the fourth numerical value and the sixth numerical value, determining that the measured object belongs to the aged person normal population, and in this case taking the fifth numerical value that is outputted by the aged person normal measurement submodel as the systolic pressure numerical value of the measured object, and if the absolute value of the difference value between the fourth numerical value and the fifth numerical value is greater than the absolute value of the difference value between the fourth numerical value and the sixth numerical value, determining that the measured object belongs to the aged person hypertension population, and in this case taking the sixth numerical value that is outputted by the aged person hypertension measurement submodel as the systolic pressure numerical value of the measured object.

It should be noted that, in the present embodiment, the measurement models used for measuring the diastolic pressure numerical values of youth, middle-aged adult, and aged person are the same; that is, the youth diastolic pressure measurement model, the middle-aged adult diastolic pressure measurement model, and the aged person diastolic pressure measurement model are one single measurement model. Furthermore, in the present embodiment, the middle-aged adult reference measurement submodel and the aged person reference measurement submodel are one single measurement model. A person skilled in the art can understand that, in practice, because there are different modeling modes for the measurement models, the youth diastolic pressure measurement model, the middle-aged adult diastolic pressure measurement model and the aged person diastolic pressure measurement model can be different measurement models, and the middle-aged adult reference measurement submodel and the aged person reference measurement submodel can also be not the same.

It should be noted that, although the operations of the method of the present invention is described in the attached figures in a specific order, that does not require or imply that these operations must be performed following the specific order, or all of the shown operations must be performed to realize the desired result. In contrast, the steps presented in the flow chart can have a varied order. Additionally or alternatively, it is feasible to omit some steps, to combine a plurality of steps into one step, and/or to divide one step into a plurality of steps.

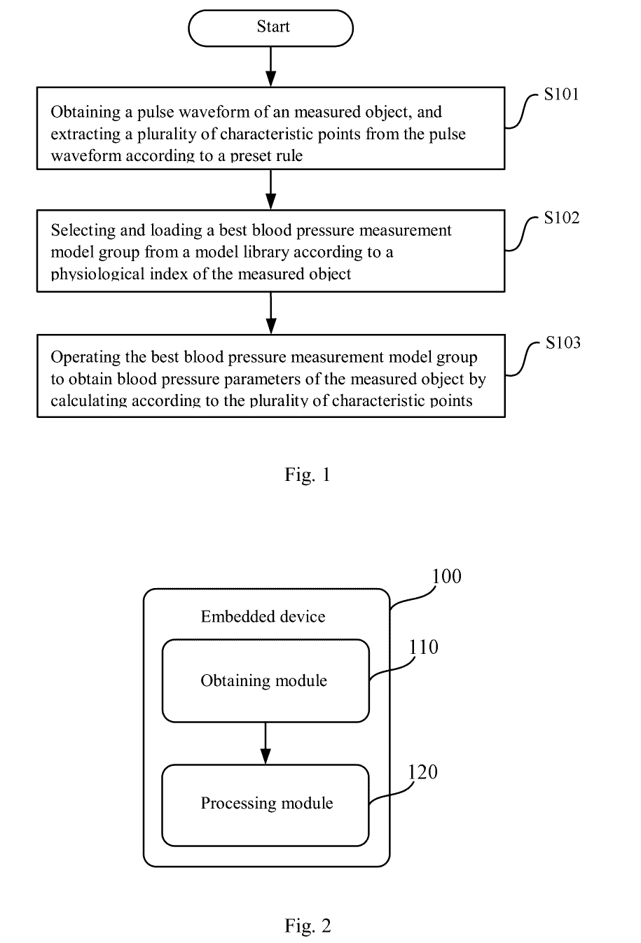

Correspondingly, the present invention further provides an embedded device for implementing the above method for measuring blood pressure. Referring to FIG. 2, FIG. 2 is the structural schematic representation of a specific embodiment of the embedded device for implementing the method for measuring blood pressure according to the present invention. As shown in the figure, the embedded device 100 comprises:

an obtaining module 110, for obtaining a pulse waveform of an measured object; and

a processing module 120, for extracting a plurality of characteristic points from the pulse waveform according to a preset rule, selecting and loading a best blood pressure measurement model group from a model library according to a physiological index of the measured object, and operating the best blood pressure measurement model group to obtain blood pressure parameters of the measured object by calculating according to the plurality of characteristic points.

The terminologies and names that appear in the present part have consistent meanings with the same terminologies and names in the above content, such as, for example, the “characteristic points”, “physiological index”, “best blood pressure measurement model group,” and “blood pressure parameters.” Those terminologies and names and the principles of operation thereof can be described and interpreted by referring to the relevant portions in the above content, and will not be described in detail here for brevity.

It should be noted that, the embedded device is preferably integrated into portable equipment, so as to facilitate the measured object performing blood pressure measurement at any moment and at any place. More preferably, in consideration of the easiness and the stability of the wearing of the portable equipment, the portable equipment is designed as having a wrist-worn structure.

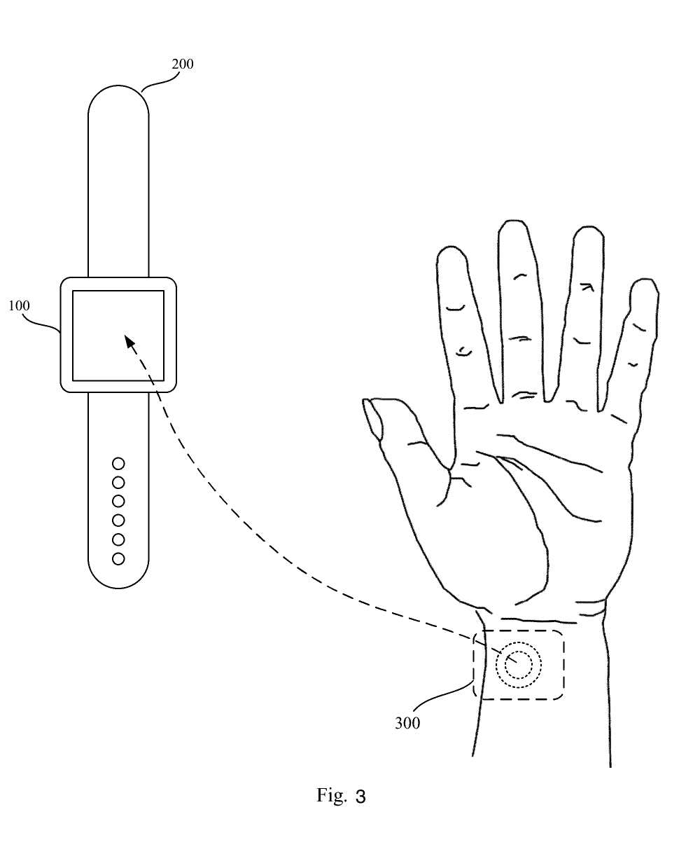

Referring to FIG. 3, FIG. 3 is the structural schematic representation of a preferable embodiment of a portable equipment that integrates the embedded device for implementing the method for measuring blood pressure and has a wrist-worn structure according to the present invention. When the portable equipment 200 shown in FIG. 3 is performing blood pressure measurement while being worn, the obtaining module 110 (not shown in FIG. 3) in the embedded device 100 is required to be placed in a location adjacent to the wrist body surface skin 300 of the measured object.

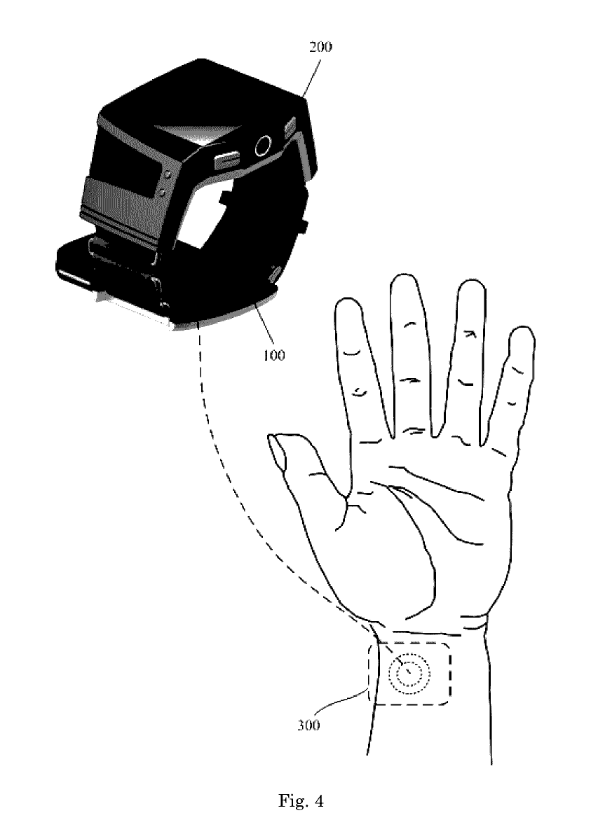

Referring to FIG. 4, FIG. 4 is the structural schematic representation of another preferable embodiment of a portable equipment that integrates the embedded device for implementing the method for measuring blood pressure and has a wrist-worn structure according to the present invention. As shown in the figure, the portable equipment 200 is an intelligent watch, that is, the embedded device 100 for implementing the method for measuring blood pressure can be integrated with an intelligent watch, and by the integrating the obtaining module 110 (not shown in FIG. 4) in the embedded device 100 may be placed in a location adjacent to the wrist body surface skin 300 of the measured object. Alternatively, the watchband of the watch can be designed as being adjustable, and the measured object can cause the obtaining module 110 to locate in a location adjacent to the wrist body surface skin 300 of the measured object by adjusting the watchband of the watch.

It should be in particular pointed out that, the wrist-worn structure of the portable equipment 200 shown in FIGS. 3 and 4 are merely illustrative, and will not limit the special appearance of the portable equipment.

For a person skilled in the art, apparently the present invention is not limited to the details of the above exemplary embodiments, and the present invention can be implemented in other concrete forms without departing from the spirit or basic features of the present invention. Therefore, in every respect, the embodiments should be deemed as exemplary and non-limiting. The scope of the present invention is defined by the attached claims rather than the above description, and therefore all the alterations that fall into the concepts and ranges of the equivalent elements of the claims are intended to be included in the present invention. Any reference elements in the claims should not be deemed as limiting the involved claims. Furthermore, as used herein, the term “comprise” does not exclude other components, units or steps, and a singular term does not exclude a plural term. The plurality of components, units or devices presented in the claims of the system can be implemented by one component, unit or device via software or hardware.

The method for measuring blood pressure and the embedded device for implementing the method provided by the present invention have the following advantages:

First, compared with the conventional mode of measuring blood pressure by using a pressure sphygmomanometer, the present invention measures blood pressure by using the characteristic points of the pulse waveform, which does not disturb human body and can realize the continuous measurement on blood pressure parameters. Compared with the conventional mode of measuring blood pressure by using pulse conducting duration, the present invention measures blood pressure by using the characteristic points of the pulse waveform, which can obtain the blood pressure parameters of the measured object that are more precise.
Second, the method, according to the physiological index of the measured object, correspondingly selects the best blood pressure measurement model group as to the measured object, whereby the accuracy of blood pressure parameter measurement can be further improved.

The above disclosures are merely some preferable embodiments of the present invention, and certainly cannot limit the scope of the claims of the present invention. Therefore, the equivalent alterations that are made according to the claims of the present invention are still within the scope of the present invention.

1. A method for measuring blood pressure, the method comprising:
obtaining a pulse waveform of a measured object;
extracting a plurality of characteristic points from the pulse waveform according to a preset rule;
selecting a best blood pressure measurement model group from a model library according to a physiological index of the measured object;
loading the best blood pressure measurement model group; and
operating the best blood pressure measurement model group to obtain blood pressure parameters of the measured object by calculating according to the plurality of characteristic points.

2. The method of claim 1, wherein the obtaining a pulse waveform of a measured object comprises:
sending a measuring light of at least one wavelength to a skin surface of the measured object;
receiving a reflected light of the measuring light; and
processing the reflected light to obtain the pulse waveform of the measured object.

3. The method of claim 2, further comprising:
providing a wrist body surface skin corresponding to a radial artery of the measured object as the skin surface.

4. The method of claim 2, wherein sending a measuring light of at least one wavelength comprises providing red light and/or infrared light as the measuring light.

5. The method of claim 4, wherein a range of a wavelength of the red light is 660 nm±3 nm.

6. The method of claim 1, wherein extracting a plurality of characteristic points from the pulse waveform comprises extracting a pulse frequency, an area of wave pattern of photoplethysmography, an area of wave pattern of principal wave upstroke, a stroke volume, a waveform factor of pulse wave, an upstroke area ratio, an average gradient of ascending limb, a relative height of dicrotic notch, and a relative height of dicrotic wave.

7. The method of claim 1, wherein selecting the best blood pressure measurement model group comprises:
determining, according to the physiological index, if the measured object is a youth, a middle-aged adult, or an aged person;
if it is determined that the measured object is a youth, selecting a youth best blood pressure measurement model group comprising a youth diastolic pressure measurement model and a youth systolic pressure measurement model;
if it is determined that the measured object is a middle-aged adult, selecting a middle-aged adult best blood pressure measurement model group comprising a middle-aged adult diastolic pressure measurement model and a middle-aged adult systolic pressure measurement model, the middle-aged adult systolic pressure measurement model comprising a middle-aged adult reference measurement submodel, a middle-aged adult normal measurement submodel and a middle-aged adult hypertension measurement submodel; and
if it is determined that the measured object is an aged person, selecting an aged person best blood pressure measurement model group comprising an aged person diastolic pressure measurement model and an aged person systolic pressure measurement model, the aged person systolic pressure measurement model comprising an aged person reference measurement submodel, an aged person normal measurement submodel and an aged person hypertension measurement submodel.

8. The method of claim 7, wherein, if the middle-aged adult best blood pressure measurement model group is selected, operating the best blood pressure measurement model group to obtain blood pressure parameters of the measured object by calculating according to the plurality of characteristic points comprises:
substituting the plurality of characteristic points into the middle-aged adult diastolic pressure measurement model and obtaining a numerical value of the diastolic pressure of the blood pressure parameters by calculating; and
substituting the plurality of characteristic points into each of the middle-aged adult reference measurement submodel, the middle-aged adult normal measurement submodel, and the middle-aged adult hypertension measurement submodel;
obtaining by calculation a first numerical value from the middle-aged adult reference measurement submodel, a second numerical value from the middle-aged adult normal measurement submodel, and a third numerical value from the middle-aged adult hypertension measurement submodel;
and selecting as a systolic pressure numerical value of the blood pressure parameters, the second numerical value or the third numerical value that is closest to the first numerical value.

9. The method of claim 7, wherein, if the aged person best blood pressure measurement model group is selected, operating the best blood pressure measurement model group to obtain blood pressure parameters of the measured object by calculating according to the plurality of characteristic points comprises:
substituting the plurality of characteristic points into the aged person diastolic pressure measurement model and obtaining a numerical value of the diastolic pressure of the blood pressure parameters by calculating; and
substituting the plurality of characteristic points into each of the aged person reference measurement submodel, the aged person normal measurement submodel, and the aged person hypertension measurement submodel;
obtaining by calculation a fourth numerical value from the aged person reference measurement submodel, a fifth numerical value from the aged person adult normal measurement submodel, and a sixth numerical value from the aged person hypertension measurement submodel;
and selecting as a systolic pressure numerical value of the blood pressure parameters, the fifth numerical value or the sixth numerical value that is closest to the fourth numerical value.

10. The method of claim 1, wherein the best blood pressure measurement model group comprises a regression equation, the method further comprising:
generating a regression coefficient of the regression equation according to a statistical treatment regarding a sample set.

11. (canceled)

12. (canceled)

13. The method of claim 4, wherein a range of a wavelength of the infrared light is 940 nm±10 nm.

14. An embedded device for measuring blood pressure of a measured object, the embedded device comprising:
an obtaining module, the obtaining module configured to perform a step of obtaining the pulse waveform of the measured object; and
a processing module, the processing module configured to perform steps of:
extracting a plurality of characteristic points from the pulse waveform according to a preset rule;
selecting a best blood pressure measurement model group from a model library according to a physiological index of the measured object;
loading the best blood pressure measurement model group; and
operating the best blood pressure measurement model group to obtain blood pressure parameters of the measured object by calculating according to the plurality of characteristic points.

15. The embedded device of claim 14, wherein the embedded device is integrated into portable equipment that includes a watchband.

16. The embedded device of claim 14, wherein obtaining the pulse waveform of the measured object comprises:
sending a measuring light of at least one wavelength to a skin surface of the measured object;
receiving a reflected light of the measuring light; and
processing the reflected light to obtain the pulse waveform of the measured object.

17. The embedded device of claim 16, wherein sending a measuring light of at least one wavelength further comprises providing red light of 660 nm±3 nm as the measuring light.

18. The embedded device of claim 16, wherein sending a measuring light of at least one wavelength further comprises providing infrared light of 940 nm±10 nm as the measuring light

19. The embedded device of claim 14, wherein extracting a plurality of characteristic points from the pulse waveform comprises extracting a pulse frequency, an area of wave pattern of photoplethysmography, an area of wave pattern of principal wave upstroke, a stroke volume, a waveform factor of pulse wave, an upstroke area ratio, an average gradient of ascending limb, a relative height of dicrotic notch, and a relative height of dicrotic wave.

20. The embedded device of claim 14, wherein selecting the best blood pressure measurement model group comprises:
determining, according to the physiological index, if the measured object is a youth, a middle-aged adult, or an aged person;
if it is determined that the measured object is a youth, selecting a youth best blood pressure measurement model group comprising a youth diastolic pressure measurement model and a youth systolic pressure measurement model;
if it is determined that the measured object is a middle-aged adult, selecting a middle-aged adult best blood pressure measurement model group comprising a middle-aged adult diastolic pressure measurement model and a middle-aged adult systolic pressure measurement model, the middle-aged adult systolic pressure measurement model comprising a middle-aged adult reference measurement submodel, a middle-aged adult normal measurement submodel, and a middle-aged adult hypertension measurement submodel; and
if it is determined that the measured object is an aged person, selecting an aged person best blood pressure measurement model group comprising an aged person diastolic pressure measurement model and an aged person systolic pressure measurement model, the aged person systolic pressure measurement model comprising an aged person reference measurement submodel, an aged person normal measurement submodel and an aged person hypertension measurement submodel.

21. The embedded device of claim 20, wherein, if the middle-aged adult best blood pressure measurement model group is selected, operating the best blood pressure measurement model group to obtain blood pressure parameters of the measured object by calculating according to the plurality of characteristic points comprises:
substituting the plurality of characteristic points into the middle-aged adult diastolic pressure measurement model and obtaining a numerical value of the diastolic pressure of the blood pressure parameters by calculating; and
substituting the plurality of characteristic points into each of the middle-aged adult reference measurement submodel, the middle-aged adult normal measurement submodel, and the middle-aged adult hypertension measurement submodel;
obtaining by calculation a first numerical value from the middle-aged adult reference measurement submodel, a second numerical value from the middle-aged adult normal measurement submodel, and a third numerical value from the middle-aged adult hypertension measurement submodel;
and selecting as a systolic pressure numerical value of the blood pressure parameters, the second numerical value or the third numerical value that is closest to the first numerical value.

22. The embedded device of claim 20, wherein, if the aged person best blood pressure measurement model group is selected, -operating the best blood pressure measurement model group comprises:
substituting the plurality of characteristic points into the aged person diastolic pressure measurement model and obtaining a numerical value of the diastolic pressure of the blood pressure parameters by calculating; and
substituting the plurality of characteristic points into each of the aged person reference measurement submodel, the aged person normal measurement submodel, and the aged person hypertension measurement submodel;
obtaining by calculation a fourth numerical value from the aged person reference measurement submodel, a fifth numerical value from the aged person adult normal measurement submodel, and a sixth numerical value from the aged person hypertension measurement submodel;
and selecting as a systolic pressure numerical value of the blood pressure parameters, the fifth numerical value or the sixth numerical value that is closest to the fourth numerical value.
